(19) United States
(12) Patent Application Publication
(10) Pub. No.: US 2023/0061204 A1
(43) Pub. Date: Mar. 2, 2023

(54) PREPARATION OF AN IMPLANTED MEDICAL DEVICE FOR A MAGNETIC RESONANCE IMAGING SCAN

(71) Applicant: MEDTRONIC, INC., Minneapolis, MN (US)

(72) Inventors: Jonathan D. Edmonson, Blaine, MN (US); Apurv Agarwal, Plymouth, MN (US); Helen W. Vogl, Shoreview, MN (US); Henry Mei, Maple Grove, MN (US); Diva U. Bhattarai, North Oaks, MN (US)

(21) Appl. No.: 17/882,214

(22) Filed: Aug. 5, 2022

Related U.S. Application Data
(60) Provisional application No. 63/237,512, filed on Aug. 26, 2021.

(51) Int. Cl. A61N 1/08; A61N 1/372

(52) U.S. Cl. CPC A61N 1/086; A61N 1/37223; A61N 1/37247

(57) ABSTRACT

An implanted medical device is prepared for a magnetic resonance imaging (MRI) scan by being programmed into an MRI mode when deemed appropriate by an external device implementing an MRI mode control application. An MRI technologist or other user may use the external device to screen the patient and implanted medical device for the MRI scan and enable the MRI mode at the implanted medical device when it is deemed appropriate in the MRI mode control application. Therapy parameters for the MRI mode may be determined on the basis of information about the device and patient, and those therapy parameters may be programmed into the implanted medical device upon enabling the MRI mode. The MRI technologist or other user may use the external device to disable the MRI mode and return to normal operation once the MRI scan is complete.

### RELATED APPLICATIONS

The present application claims priority to U.S. Provisional Application No. 63/237512, filed on Aug. 26, 2021, which is incorporated by reference herein.

### TECHNICAL FIELD

Embodiments provide methods, devices, and systems that prepare an implanted medical device for a magnetic resonance imaging (MRI) scan.

### BACKGROUND

Implanted medical devices implanted within a patient are exposed to magnetic fields as well as radio frequency electromagnetic energy when the patient undergoes an MRI scan. The conditions can cause problems for the implanted medical device of a patient during the MRI. For instance, an implanted cardiac device that relies on measuring heart rate to provide appropriate cardiac therapy may be affected by a fluctuating magnetic field from the MRI scan that may cause an incorrect hear rate measurement.

To address the issues that may result from the conditions during an MRI scan, the implanted medical device may be programmed into an MRI mode of operation, such as the Medtronic SureScan™ mode. The MRI mode may alter the operations of the implanted medical device to provide therapy that is acceptable during the MRI scan while being less susceptible to the MRI scan conditions. For instance, a cardiac device may enter an asynchronous form of pacing at a fixed pacing rate so that a heart rate measurement is not needed to set the pacing rate.

While the MRI mode can be an effective way to address the conditions of the MRI scan, the implanted medical device must enter the MRI mode at some point before the MRI scan and exit the MRI mode at some point after the MRI scan. This requires that the implanted medical device have such an MRI mode capability and then requires that the implanted medical device be programmatically switched to the MRI mode. However, because the MRI mode is an altered form of therapy, the switch to the MRI mode should occur only when the medical status of the patient with the implanted medical device allows.

In present workflows, to confirm that switching to the MRI mode is possible for the device and appropriate for the patient, a cumbersome process must be followed that requires the involvement of someone experienced with the operation of the implanted device. It would be beneficial for personnel working in the MRI environment to have the capability of executing this process, which could be carried out by MRI technologists, radiographers or radiology nursing staff. However, radiology personnel may not be equipped to adequately determine the medical status of the patient and may not be equipped to determine that the implanted medical device has an MRI mode available. Additionally, the MRI technologist and others are incapable of instructing the implanted medical device to enter the MRI mode. Therefore, an MRI is a significantly more cumbersome process for a patient with an implanted medical device.

The patient must first visit a clinician who verifies that the medical status of the patient allows for use of the MRI mode. Next, the MRI mode capability of the implanted medical device must be confirmed such as by the clinician. Then, once it is determined that the patient can obtain the MRI scan, an implanted device technician capable of programming the implanted medical device into the MRI mode utilizing specialized equipment must be present at the time of the MRI scan to activate the MRI mode before the MRI scan and deactivate the MRI mode once the MRI scan has ended. Thus, valuable personnel resources, equipment resources, and time are required which creates added burden on the patient as well as the clinicians and technologists that are also involved.

### SUMMARY

Embodiments address issues such as these and others by providing the methods, devices, and systems that prepare the implanted medical device for the MRI scan. An MRI technologist or similar radiology personnel such as radiology nursing staff or other healthcare workers in the MR environment, can utilize these embodiments to move forward with the MRI scan without needing assistance from a clinician or device technician. These embodiments may perform device and patient determinations to confirm that both device and patient are eligible. These embodiments may also provide the communications necessary to activate and deactivate the MRI mode at the implanted medical device.

Embodiments provide a method of preparing an implanted medical device of a patient for a magnetic resonance imaging (MRI) scan that involves wirelessly communicating from an external device to the implanted medical device to begin receiving current therapy and device parameters. The method involves determining by the external device whether the therapy parameters and device parameters are within acceptable ranges for the MRI scan. When the therapy parameters and device parameters are within acceptable ranges for the MRI scan, displaying a first user selectable control at the external device to implement an MRI mode of operation at the implanted medical device. When one or more of either the therapy parameters or the device parameters are outside of the acceptable ranges for the MRI scan, displaying a message at the external device regarding not conducting the MRI scan and not displaying the first user selectable control to implement the MRI mode of operation.

Embodiments provide an external device for preparing an implanted medical device of a patient for a magnetic resonance imaging (MRI) scan. The external device comprises a display, a user interface, a wireless telemetry circuit, and a processing device. The processing device is in communication with the display, the user interface, and the wireless telemetry circuit. The processing device is configured to wirelessly communicate with the implanted medical device to begin receiving current therapy and device parameters by using the telemetry circuit. The processing device is configured to determine whether the therapy parameters and device parameters are within acceptable ranges for the MRI scan. When the therapy parameters and device parameters are within acceptable ranges for the MRI scan, the processing device is configured to display a first user selectable control on the display to implement an MRI mode of operation at the implanted medical device, the first user selectable control being selectable via the user interface. When one or more of either the therapy parameters or the device parameters are outside of the acceptable ranges for the MRI scan, the processing device is configured to display a message on the display regarding not conducting the MRI scan and not displaying the first user selectable control to implement the MRI mode of operation.

Embodiments provide a system that includes an implanted medical device and an external device for preparing the implanted medical device of a patient for a magnetic resonance imaging (MRI) scan. The external device includes a display, a user interface, a wireless telemetry circuit, and a processing device. The processing device is in communication with the display, the user interface, and the wireless telemetry circuit. The processing device is configured to wirelessly communicate with the implanted medical device to begin receiving current therapy and device parameters by using the telemetry circuit. The processing device is configured to determine whether the therapy parameters and device parameters are within acceptable ranges for the MRI scan. When the therapy parameters and device parameters are within acceptable ranges for the MRI scan, the processing device is configured to display a first user selectable control on the display to implement an MRI mode of operation at the implanted medical device, the first user selectable control being selectable via the user interface. When one or more of either the therapy parameters or the device parameters are outside of the acceptable ranges for the MRI scan, the processing device is configured to display a message on the display regarding not conducting the MRI scan and not displaying the first user selectable control to implement the MRI mode of operation.

### DESCRIPTION OF THE DRAWINGS

FIG. 1 shows an operating environment for embodiments of devices and system disclosed herein for preparing the implanted medical device for the MRI scan.

FIGS. 2A and 2B show an example of an operational flow of an MRI mode control application implemented on an external device to interact with the implanted medical device and a user in order to prepare the implanted medical device for the MRI scan.

FIGS. 3A and 3B show an example of operational flow of a heart rate tracking module implemented on the external device in conjunction with the MRI mode control application.

FIG. 4 shows an example of operational flow of a post-MRI scan pacing amplitude module implemented on the external device in conjunction with the MRI mode control application.

FIG. 5 shows an example of a display for a device look-up tool that may be implemented on the external device or a separate external device.

FIG. 6 shows an example of a first display of the MRI mode control application regarding connecting to a connector device.

FIG. 7 shows an example of a second display of the MRI mode control application regarding confirming the implanted medical device is MRI capable.

FIG. 8 shows an example of a third display of the MRI mode control application regarding connecting to the implanted medical device.

FIG. 9 shows an example of a fourth display of the MRI mode control application regarding progress of an interrogation of the implanted medical device.

FIG. 10 shows an example of a fifth display of the MRI mode control application regarding an unsupported implanted medical device.

FIG. 11 shows an example of a sixth display of the MRI mode control application regarding the MRI mode already being activated.

FIG. 12 shows an example of a seventh display of the MRI mode control application regarding a reason the MRI mode control application will not activate MRI mode.

FIG. 13 shows an example of an eighth display of the MRI mode control application regarding providing a user selectable control to activate the MRI mode.

FIG. 14 shows an example of a ninth display of the MRI mode control application regarding providing user selectable controls to deactivate the MRI mode or continue depending upon patient comfort with the activated MRI mode.

FIG. 15 shows an example of a tenth display of the MRI mode control application regarding removing the connector device.

FIG. 16 shows an example of an eleventh display of the MRI mode control application regarding the implanted medical device and patient being ready for the MRI scan.

FIG. 17 shows an example of a twelfth display of the MRI mode control application regarding reconnecting the connector device to the implanted medical device.

FIG. 18 shows an example of a thirteenth display of the MRI mode control application regarding providing a user selectable control to deactivate the MRI mode.

FIG. 19 shows an example of a fourteenth display of the MRI mode control application regarding the MRI mode being deactivated.

FIG. 20 shows an example of a fifteenth display of the MRI mode control application regarding an interruption to communication with the implanted medical device.

FIG. 21 shows an example of a first report from the MRI mode control application that may be viewed prior to activating the MRI mode.

FIG. 22 shows an example of a second report from the MRI mode control application that may be viewed after activating the MRI mode.

FIG. 23 shows an example of a third report from the MRI mode control application that may be viewed after deactivating the MRI mode.

FIG. 24 shows an example of components of the external device.

### DETAILED DESCRIPTION

Embodiments allow for the preparation of an implanted medical device for an MRI scan by communicating with the implanted medical device to perform various determinations and to allow the MRI mode to be activated on the implanted medical device. An MRI technologist or other user can rely upon the operations of an external device to perform these determinations and guide the MRI technologist or other user to activate and deactivate the MRI mode and conduct the MRI scan.

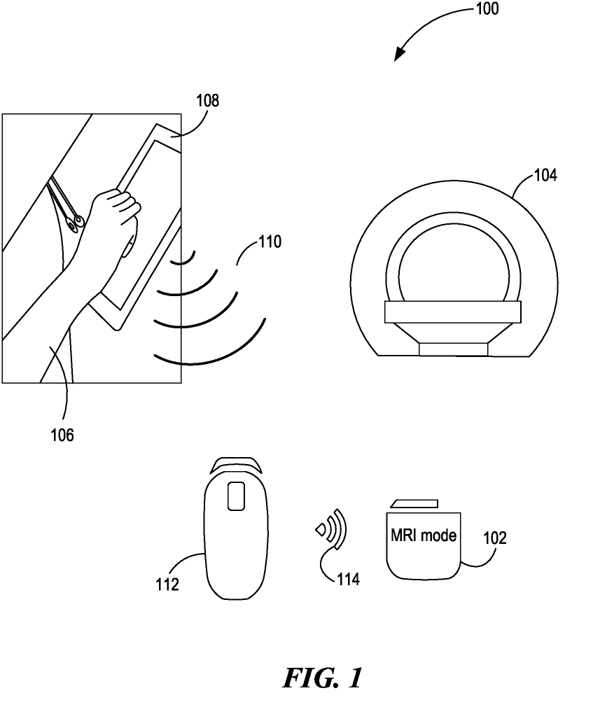

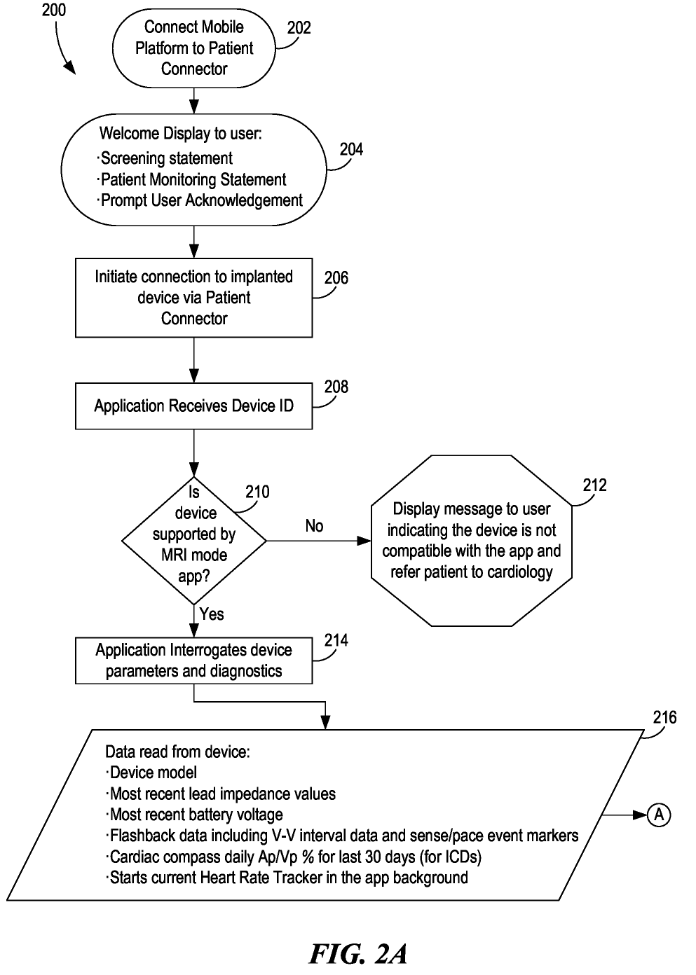

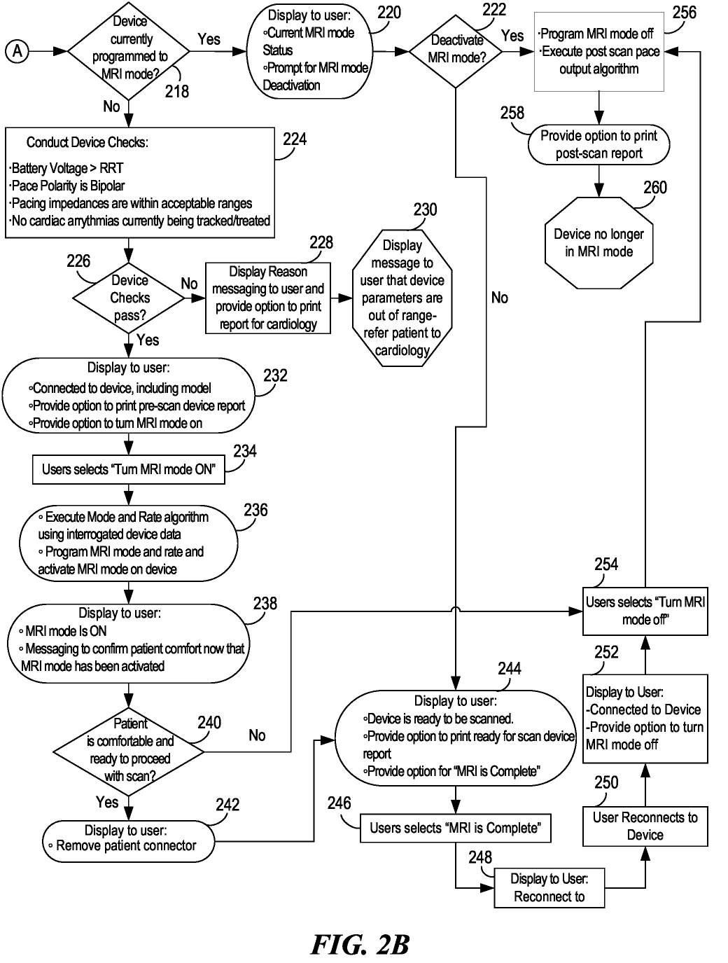

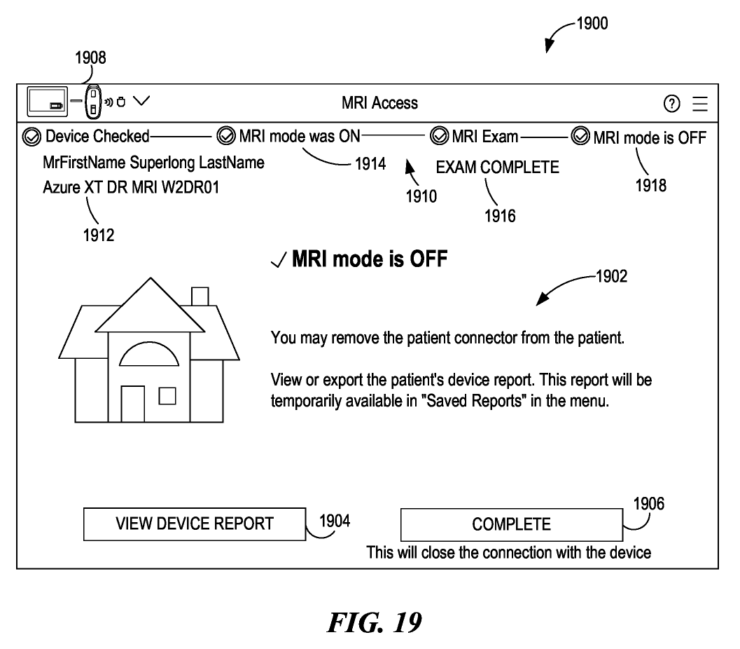

FIG. 1 shows an example of an environment 100 such as an MRI exam facility where a patient having the implanted medical device 102 is present in order to receive an MRI scan within an MRI machine 104. The MRI technologist or other user 106 is present and is using an external device 108 that guides the MRI technologist or other user 106 to prepare the implanted medical device 102 for the MRI scan prior to the patient entering the room where the MRI machine 104 is present. The external device 108 utilizes longer range wireless communications 110 to ultimately communicate with the implanted medical device 102 to exchange information and instructions where those wireless communications 110 are longer range signals such as using the Bluetooth ® protocol and/or the Medical Implant Communications System (MICS) band protocols. The components and operations of the external device 108 are discussed in more detail below in relation to FIG. 2A-24.

It will be appreciated that in some cases, the implanted medical device 102 may communicate directly with the external device 110 using the longer-range wireless communications 110 while in other cases the implanted medical device 102 lacks the ability to communicate using the longer-range signals 110. In the latter case, a connector device 112 as shown in FIG. 1 is present to exchange shorter range wireless communications 114, such as near field inductive coupling signals, with the implanted medical device 102. In this scenario, the connector device 112 exchanges the longer-range wireless communications 110 with the external device 108 and exchanges shorter range wireless communications 114 with the implanted medical device 102. In this manner, the connector device 112 passes through information and instructions between the external device 108 and the implanted medical device 102. However, this requires that the MRI technologist or other user 106 properly position the connector device 112 relative to the implanted medical device 102 to establish the shorter-range communications 114.

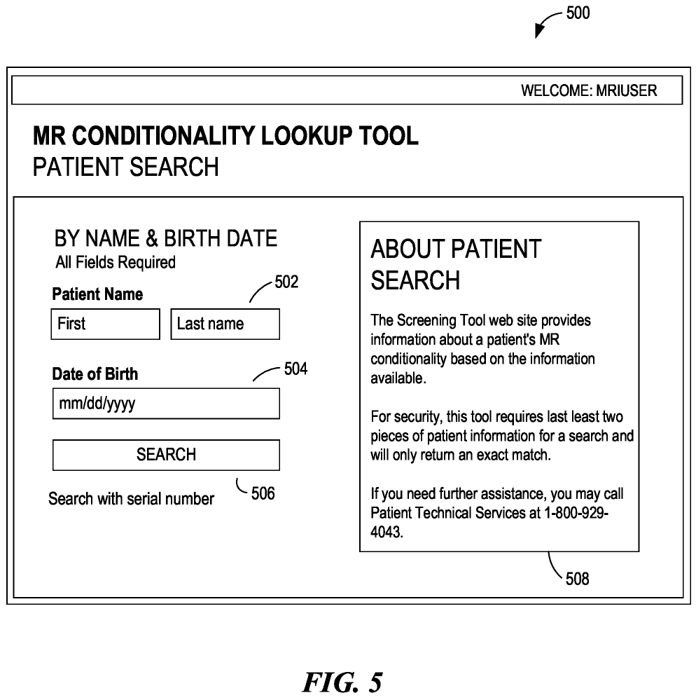

Upon the patient arriving for the MRI scan and prior to the patient entering the room where the MRI machine 104 is located, the MRI technologist or other user 106 may access one or more applications of the external device 108 to facilitate the preparation of the implanted medical device 102 for the MRI scan. Embodiments may provide multiple tools to assist the MRI technologist or other user 106. A device look-up tool, which may be implemented either on the external device 108 or a separate computer device unrelated to communications with the implanted medical device 102, allows the MRI technologist or other user 106 to look-up the patient to determine if the implanted medical system of that patient, is MRI compatible and under what conditions. This determination may include considering the MRI conditionality of the implanted medical device 102 and any other components of the implanted medical system including any implanted medical leads. The device look-up tool may be implemented as dedicated application on the external device 108 or other computer or may be a website or other network provided resource available through a web browser or similar multi-purpose application on the external device 108 or other computer. An example of a display screen 500 provided to the MRI technologist or other user 106 is shown in FIG. 5. This tool allows the MRI technologist or other user to enter the name of the patient in the data fields 502 as well as the date of birth of the patient in data field 504 with a user selectable search control in the form of button 506 to start the search. Information 508 is displayed to further inform the MRI technologist or other user 106.

Upon selecting the search button 506, the tool performs a look-up in a database to return a result that the components of the implanted medical system, including the implanted medical device 102 and any implanted leads of the patient, is or is not MRI compatible. The database associates at least the patient name, date of birth, and the indication of whether the implanted medical device 102, implanted leads, and any other implanted components of the patient is or is not MRI compatible and what the conditions for MRI compatibility may be. For instance, the implanted medical device 102 may be compatible with certain conditions such as the strength of the magnetic fields and/or RF electromagnetic energy of the MRI machine 104. The MRI technologist or other user 106 may then ensure that the MRI machine 104 and the settings used for the MRI scan are appropriate for the implanted medical device 102, implanted leads, and any other components of the implanted medical system.

The database of this information accessed by the device look-up tool may be in various locations. For instance, the database may be located at a network location, such as at a physical location remote from the location of the MRI scan so that the database is accessed over a wide area network like the Internet. For instance, the manufacturer of the implanted medical device 102 may maintain such a database that is made available to the device look-up tools made available to MRI technologist or other user 106. Alternatively, the database may be stored at a local network location at the location of the MRI scan or within the storage of the external device 108.

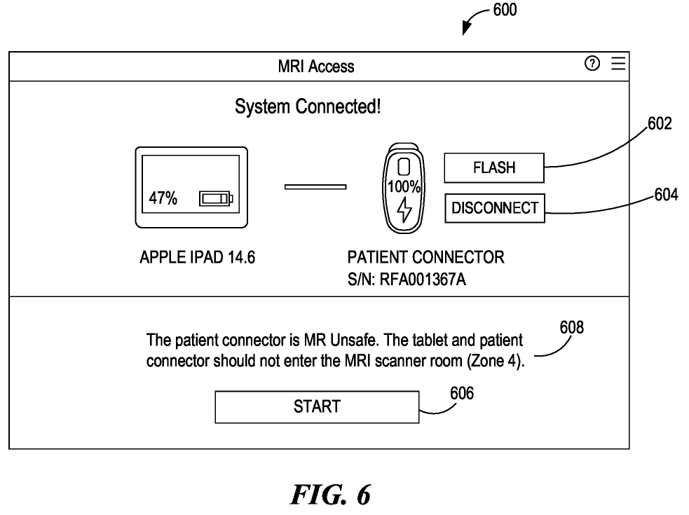

The external device 108 also has an MRI mode control application that guides the MRI technologist or other user during the process of determining that the MRI mode is appropriate and then activating the MRI mode of the implanted medical device 102. FIGS. 2A and 2B show one example 200 of operations of an MRI mode control application while FIGS. 6-20 show examples of display screens that are shown to the user on the display of the external device 108 during the operations of the MRI mode control application. The MRI mode control application may also be implemented as a dedicated application on the external device 108 or may be a website or other network provided resource available through a web browser or similar multi-purpose application on the external device 108 which produces a display on the external device that has user selectable controls for proceeding with operation of the application.

Upon the patient arriving at the MRI facility and the MRI technologist or other user 106 accessing the MRI mode control application of the external device 108, the MRI mode control application then proceeds with operations as shown in FIGS. 2A and 2B. In the scenario where a connector device 112 is being used, the MRI control mode application awaits the connection of the external device 108 to the connector device 112 at an operation 202 which results in the display screen 600 of FIG. 6 upon the connection occurring.

The display screen 600 displays options and information for the MRI technologist or other user 106. A user selectable control 602 allows the MRI technologist or other user 106 to conduct a flash which may enable an indicator light of the connected connector device 112. This allows the MRI technologist or other user 106 to confirm that the MRI mode control application is communicating with the correct connector device 112, which may be relevant in an environment where multiple connector devices 112 are nearby. A user selectable control 604 allows the MRI technologist or other user 106 to disconnect the external device 108 from the connector device 112 if the connection is no longer needed due to a delay or cancellation of the MRI scan. Information 608 warns the MRI technologist or other user 106 regarding the connector device 112 not being MRI compatible and therefore should not enter the room where the MRI machine 104 is located. A user selectable control 606 is present and allows the MRI technologist or other user 106 to select the control 606 to then proceed with the operations of the MRI mode control application.

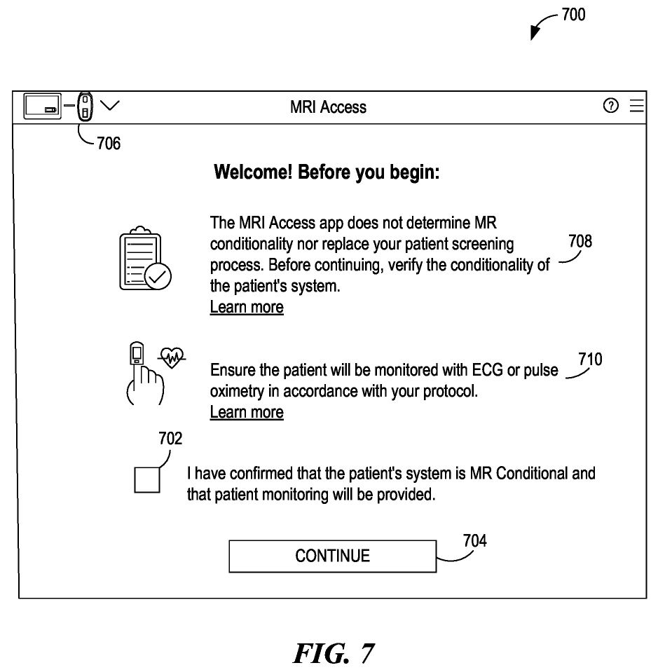

Upon selecting control 606, operation 204 then results in a welcome display screen 700 of FIG. 7 being displayed at the external device 108. Information 708 explains that the MRI technologist or other user 106 should verify the MRI compatibility, such as by using the device look-up tool previously discussed, which may be expressed in terms of conditionality as discussed above in relation to FIG. 5. Information 710 explains that the patient should be monitored with electrocardiography (ECG) or pulse oximetry per the MRI technologist’s or other user’s existing protocols. Display screen 700 also provides a connection status indicator 706 regarding the connection of the external device 108 to other devices such as the connector device 112 as shown. This screen 700 also provides a user selectable control 702 to indicate to the MRI mode control application that the MRI technologist or other user 106 has confirmed that the implanted medical device 102 is MRI conditionally compatible and that the MRI technologist or other user 106 will be monitoring the patient during the procedure. Once the user selectable control 702 is selected, then another user selectable control in the form of button 704 becomes active and available for selection by the user.

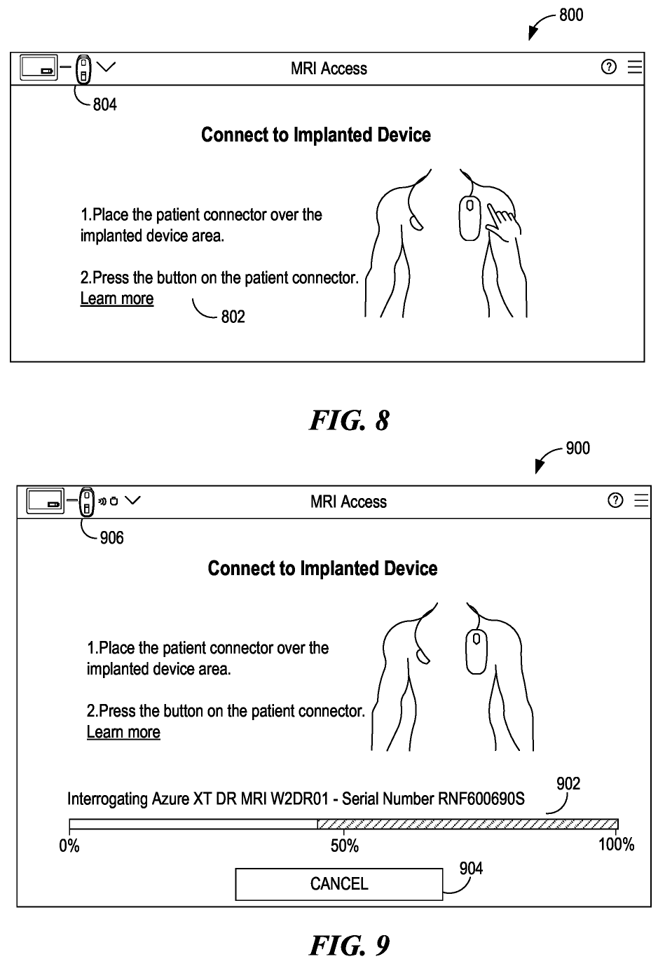

Upon selecting the control 704, operation 206 results in the display of an instructional display screen 800 of FIG. 8 that includes information 802 that instructs the MRI technologist or other user 106 to place the connector device 112 over the area of the implanted medical device 102. This instructed placement of the connector device 112 allows the external device 108 to begin communicating with the implanted medical device 102 in the scenario where the implanted medical device 102 lacks the ability to communicate using the longer-range signals 110. A connection status indicator 804 again shows the connection status of the external device 108.

Upon the MRI technologist or other user 106 placing the connector device 112 over the implanted medical device 102 so that the MRI mode control application detects the ability to communicate with the implanted medical device 102, operation 208 begins communicating with the implanted medical device 102. The external device 108 obtains a device identifier from the implanted medical device 102 that allows the MRI mode control application to determine whether the implanted medical device is compatible with the function of the MRI mode control application at a query operation 210. While some MRI compatible implanted medical devices may be capable of being switched into the MRI mode by the MRI mode control application, other MRI compatible implanted medical devices may not. The MRI mode control application may be manufacturer/brand agnostic and therefore be applicable to implanted devices from many manufacturers and brands, so the reason for the implanted device not being compatible with MRI mode control application may be unrelated to the manufacturer and brand but may be related to other considerations such as the particular technical capabilities of the implanted medical device. The MRI mode control application performs a look-up of the device identifier within a database at the query 210 to make the determination, where the database may be locally stored at the external device 108 or may be accessible over a local or wide area network connection.

When the MRI mode control application determines that the implanted medical device is compatible with the MRI mode control application, then operation 214 begins interrogating the implanted medical device 102 to obtain both current device and therapy parameters. Operation 214 also results in a progress display screen 900 of FIG. 9 to be shown at the external device 108. The display screen 900 is similar to the display screen 800 but adds a progress bar 902 to show the progress of the interrogation of the implanted medical device 102 and also includes a user selectable cancel button 904 to stop the interrogation. The connection status indicator 906 is also shown and now shows that the connection status is the external device connected to the connector device 112 and to the implanted medical device 102.

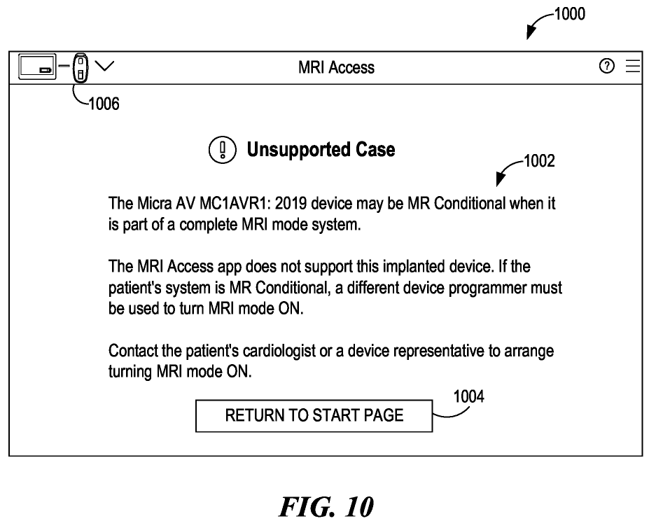

Returning to query operation 210, when it is determined that the implanted medical device is not compatible with the MRI mode control application, terminating status 212 occurs which results in a termination display screen 1000 of FIG. 10. Display screen 1000 includes information 1002 explaining that the implanted medical device 102 is not compatible with the MRI mode control application. A user selectable control button 1004 allows the user to select to return to the start page. The connection status indicator 1006 now shows that the connector device 112 has disconnected from the implanted medical device 102.

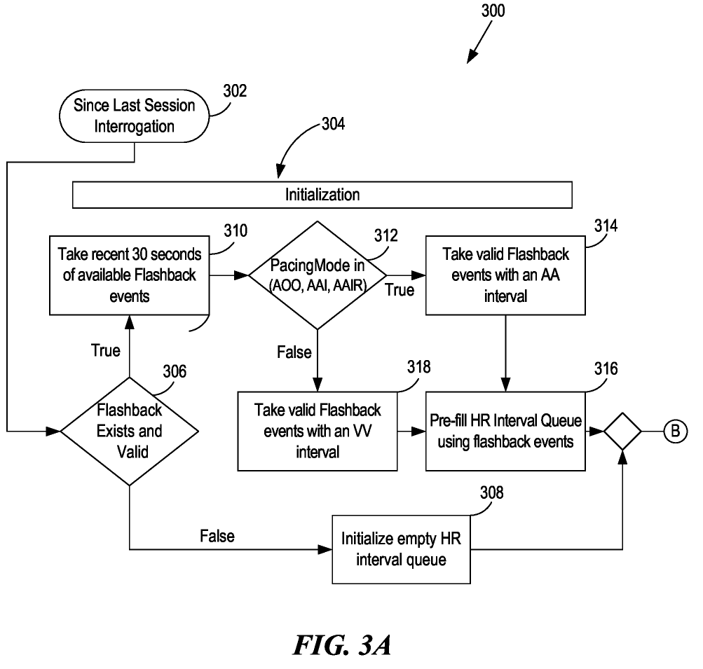

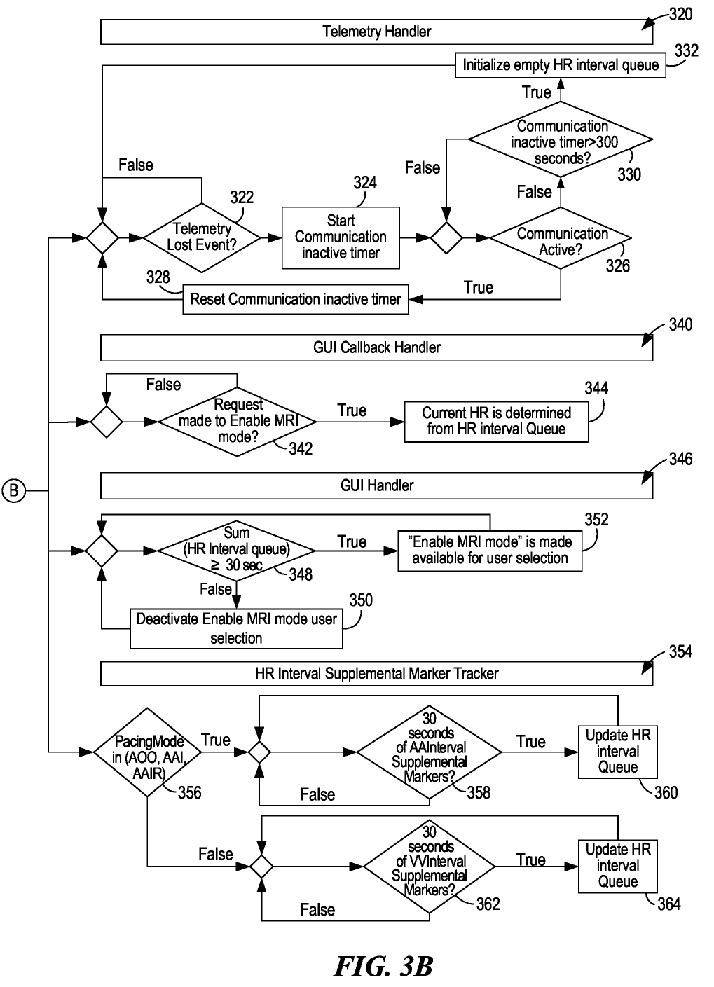

Returning to the operation 214, the interrogation completes at a state 216 where the MRI mode control application now has the data from the device that is necessary to proceed with the preparation of the implanted medical device 102. Examples of the information include the device model, impedance values for leads connected to the implanted medical device 102, battery voltage, flashback data that is stored data with recent ventricular-to-ventricular (V-V) intervals and sense/pace event markers in the case of a cardiac therapy device, daily Ap/Vp percentages for the last 30 days in the case of an implanted cardiac defibrillator device. Additionally, at this point the MRI mode control application may initiate a heart rate tracker to run in the background. The operations of the heart rate tracker are discussed in more detail below with reference to FIGS. 3A and 3B.

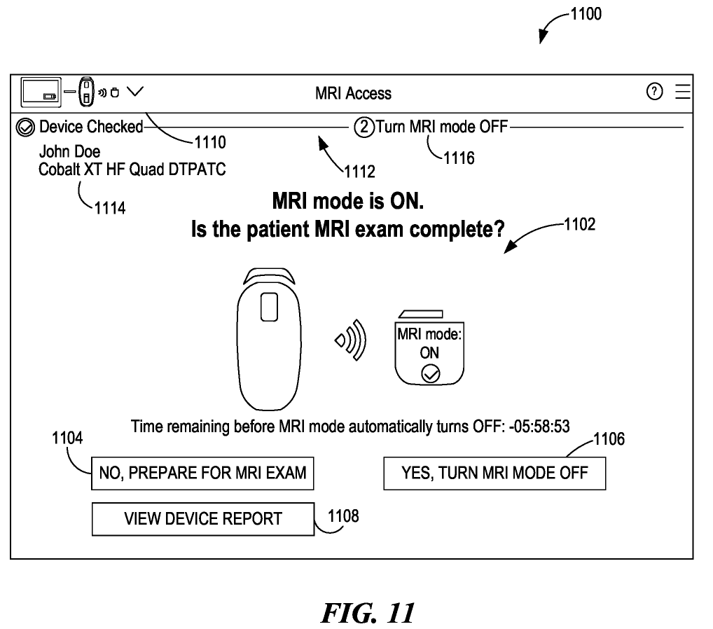

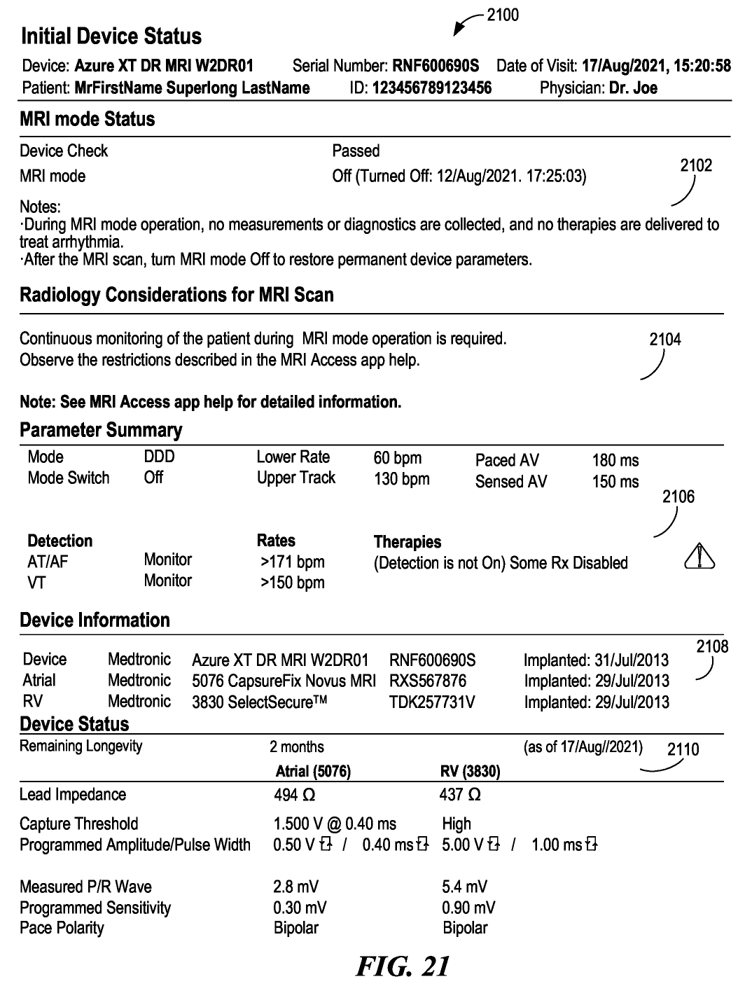

The MRI mode control application then determines whether the implanted medical device 102 is already in the MRI mode at query operation 218 based on the mode of operation obtained during the interrogation. If the implanted medical device 102 is already in the MRI mode, then operation 220 results in a display screen 1100 of FIG. 11 being displayed at the external device. Display screen 1100 includes information 1102 showing that the MRI mode is already active and includes a procedural progression timeline 1112 showing that the implanted medical device 102 has been checked at stage 1114 and waiting for MRI mode to be deactivated at stage 1116. Display screen 1100 also provides user selectable control 1104 to allow the MRI technologist or other user 106 to select to maintain the MRI mode to prepare for the MRI. Display screen 1100 also provides user selectable control 1106 to allow the MRI technologist or other user 106 to select to deactivate the MRI mode should the MRI technologist or other user 106 decide that the MRI will not be conducted. At this time, the MRI technologist or other user 106 can also choose to view a device report by selecting a user selectable control 1108. An example of such a report is shown in FIG. 21 and discussed in more detail below.

Query 222 determines whether the MRI technologist or other user 106 has selected to maintain MRI mode or to deactivate MRI mode. When the MRI technologist or other user selects the user selectable control 1104 to continue preparing for the MRI scan, the process continues to operation 244 which is discussed in more detail below. When the MRI technologist or other user selects the user selectable control 1106 to deactivate the MRI mode, the process continues to operation 256 also discussed in more detail below.

Returning to query operation 218, when it is determined that the implanted medical device 102 is not already in the MRI mode, the process continues to operation 224 where the MRI mode control application performs checks of various device and therapy parameters obtained from the implanted medical device 102 against acceptable device and therapy parameter ranges involving thresholds or requirements. For instance, the MRI mode control application of this example checks whether the battery level is greater than a threshold such as a recommended replacement time (RRT) voltage for this implanted medical device 102. The MRI mode control application of this example checks whether a pace polarity, in the case of a pacing device, is bipolar. The MRI mode control application of this example checks whether pacing impedances through the pacing leads attached to the implanted medical device 102 are within acceptable ranges. The MRI mode control application of this example checks whether any cardiac arrhythmias are currently present and/or currently being treated.

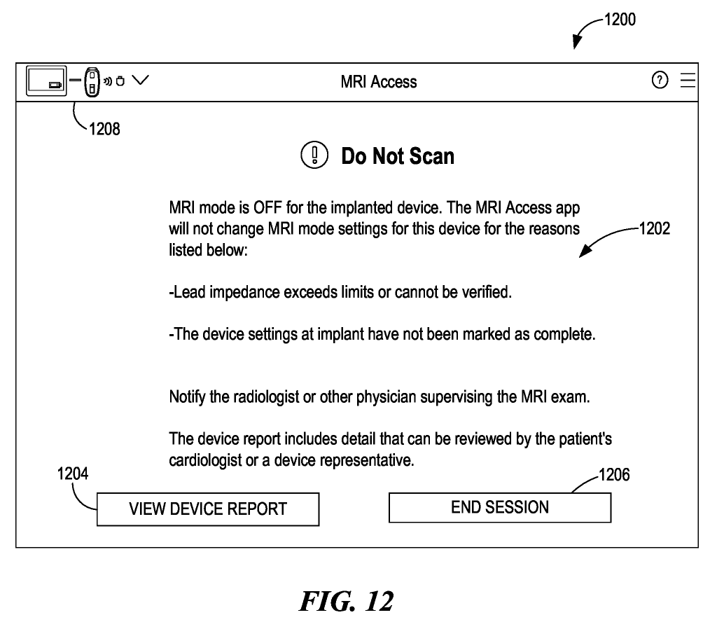

A query operation 226 detects whether all checks of the current device and therapy parameters have passed by falling within acceptable ranges. If one or more checks fail, then the process continues to operation 228 and termination state 230 where a display screen 1200 of FIG. 12 is displayed to indicate to the MRI technologist or other user 106 that the MRI scan cannot be performed. The display screen 1200 includes information 1202 that explains which checks have failed. A user selectable control 1204 allows the user to view a pre-scan device report such as the report in FIG. 21 while user selectable control 1206 allows the user to end the session with the implanted medical device 102. The connection status indicator 1208 is present and shows that the external device 108 is currently connected to the connector device 112 and implanted medical device 102.

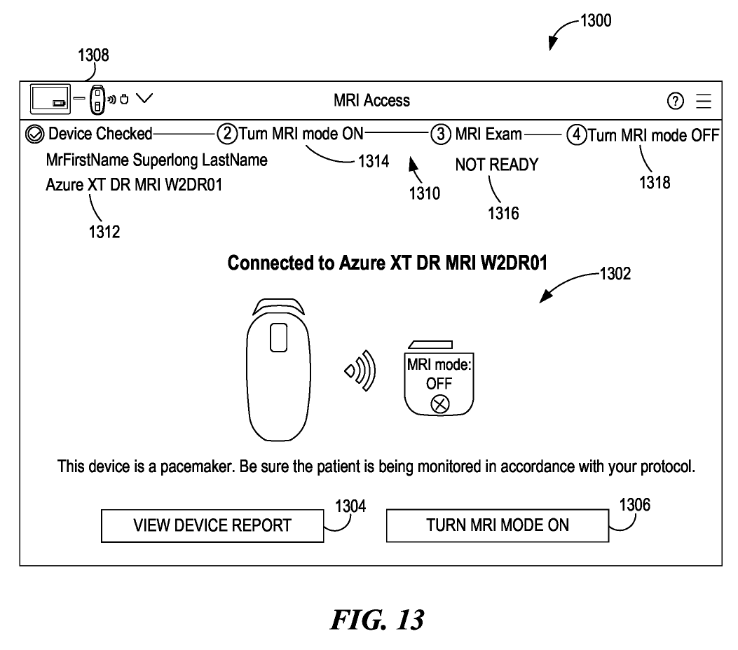

Returning to query operation 226, if all checks have passed, then the process continues to operation 232 which results in a display screen 1300 of FIG. 13 being displayed at the external device 108. Display screen 1300 shows a connected status 1302 to the implanted medical device 102 and includes a user selectable control 1304 to generate a pre-scan report as shown in FIG. 21. Display screen 1300 also includes the connection status indicator 1308 as well as the procedural progression timeline 1310 showing that the implanted medical device 102 has been checked at stage 1312, waiting for MRI mode to be activated at stage 1314, with the device not being ready for the MRI scan at stage 1316 or the deactivation of MRI mode at stage 1318 since MRI mode has not yet been activated. Display screen 1300 also includes a user selectable control 1306 that provides the MRI technologist or other user 106 with the ability to activate MRI mode.

It should be noted that the user selectable control 1306 has not been displayed in any prior display screens, as display screen 1300 from operation 232 is the first occurrence of a user selectable control for activating the MRI mode. Therefore, the MRI technologist or other user 106 has had no opportunity to inadvertently place the implanted medical device 102 into the MRI mode prior to the implanted medical device 102 being successfully interrogated for information and the device and therapy parameters from the implanted medical device 102 satisfactorily passing the checks. In this manner, the MRI mode control application has successfully guided the MRI technologist or other user 106 through the process of verifying that the implanted medical device 102 is ready for activation of the MRI mode.

Upon receiving the selection of the control 1306 at operation 234, operation 236 then initiates a mode and rate algorithm to determine therapy parameters for the MRI mode including the proper pacing mode and pacing rate in the case of an implanted cardiac device. Such mode and rate algorithms are known in the art, such as those disclosed in U.S. Pat. No. 10,293,167 and U.S. Pat. No. 10,441,798, both of which are incorporated by reference herein. The determination of the therapy parameters for the MRI mode may be based on the current therapy parameters obtained from the implanted medical device 102. Also at operation 236, the external device sends an instruction to the implanted medical device 102 to activate the MRI mode.

In one scenario, the external device 108 performs the pacing mode and rate determination based on information that has been received from the implanted medical device 102 by performing the mode and rate algorithm. In that case, once the pacing mode and rate for the MRI mode are determined, the external device 108 then sends and instruction to enter the MRI mode and programs the determined pacing mode and rate for the MRI mode at the implanted medical device 102 so that the implanted medical device 102 may then implement the MRI mode with the determined pacing mode and rate. In another scenario, the implanted medical device 102 may be configured to do the mode and rate determination upon receiving an instruction to enter the MRI mode. In that case, the external device 108 submits the instruction to enter the MRI mode and then relies on the implanted medical device to determine the proper pacing mode and rate to use during the MRI mode.

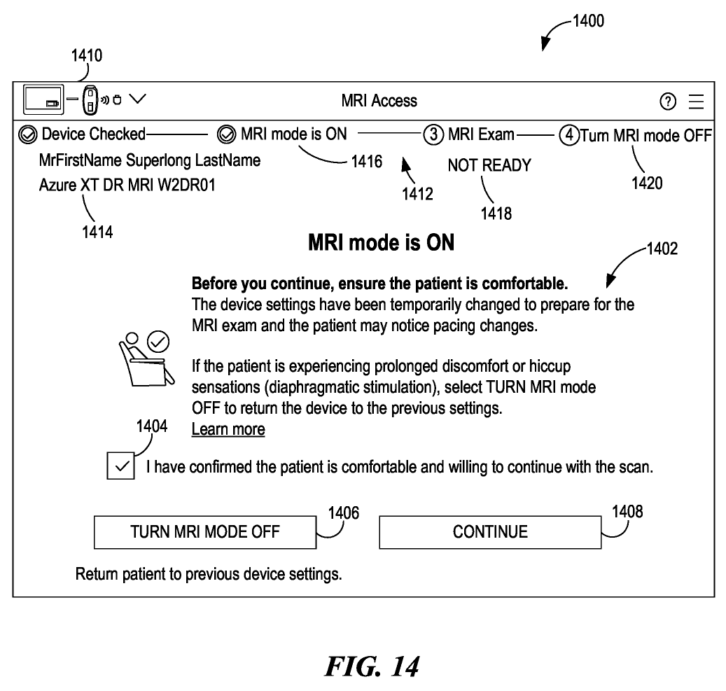

At this point, the process continues to operation 238 that results in the display of a display screen 1400 of FIG. 14. The display screen 1400 includes information 1402 explaining that the MRI mode is active and includes a prompt that the MRI technologist or other user 106 should confirm that the patient is comfortable with MRI mode being active. A user selectable control 1404 allows the MRI technologist or other user 106 to confirm that the patient is comfortable. If the patient is not comfortable, then the MRI technologist or other user 106 may deactivate the MRI mode by selecting a use selectable control 1406. However, if the patient is comfortable, then upon the MRI technologist or other user selecting the control 1404, a user selectable control 1408 becomes available to allow the MRI technologist or other user 106 to select the control 1408 and continue the process of preparing for the MRI scan.

The display screen 1400 includes additional information. The connection status indicator 1410 shows the connection of the external device 108 to the connector device 112 and implanted medical device 102. The procedural progression timeline 1412 is also present to show that the implanted medical device 102 has been checked at stage 1414, the MRI mode is activated at stage 1416, with the device not being ready for the MRI scan at stage 1418 or the deactivation of MRI mode at stage 1420 since the connector device 112 has not been removed and the MRI scan has not yet been performed.

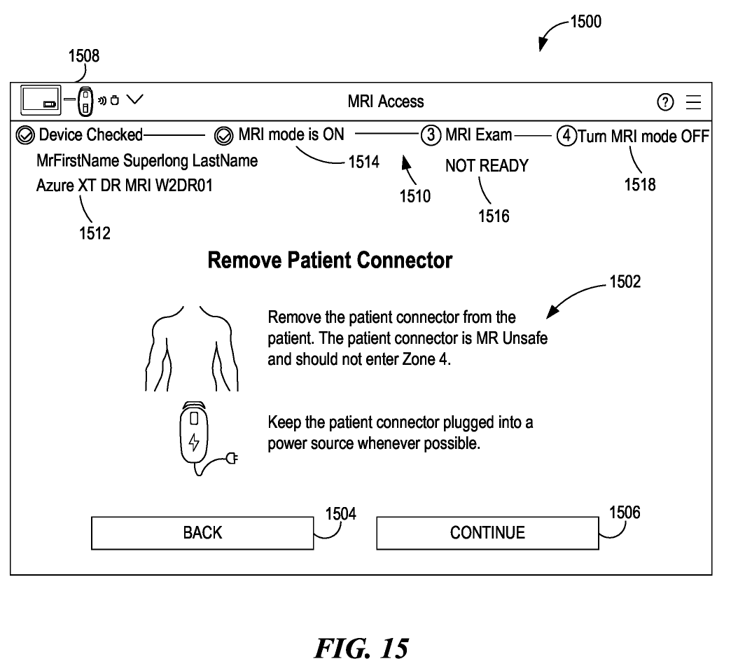

A query operation 240 detects whether the MRI technologist or other user 106 selected the control 1406 or the control 1408. If the control 1406 is selected to turn off the MRI mode as at operation 254, then the process continues to operation 256 which is discussed in more detail below. If the control 1408 is selected to continue, then the process continues to operation 242 which results in the display of a display screen 1500 of FIG. 15. The display screen 1500 includes information 1502 indicating that the MRI technologist or other user 106 should remove the connector device 112 from the patient since the connector device 112 should not accompany the patient into the room with the MRI machine 104.

The display screen 1500 includes additional features such as a user selectable control 1504. This control 1504 allows the MRI technologist or other user to move back to the display screen 1400, for instance to turn off the MRI mode if the patient has become uncomfortable. The display screen 1500 includes a user selectable control 1506 that allows the process to continue. The connection status indicator 1508 shows the connection of the external device 108 to the connector device 112 and implanted medical device 102. The procedural progression timeline 1510 is also present to show that the implanted medical device 102 has been checked at stage 1512, the MRI mode is activated at stage 1514, with the device not being ready for the MRI scan at stage 1516 or the deactivation of MRI mode at stage 1518 since the connector device 112 has not been removed and the MRI mode has not yet been performed.

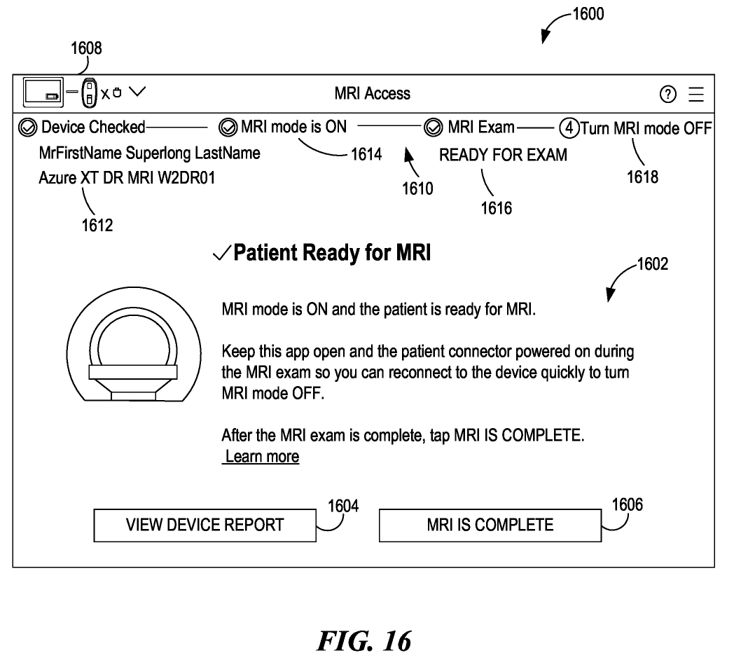

Upon selecting control 1506 once the connector device 112 has been removed, the process continues to operation 244 which results in a display screen 1600 of FIG. 16 being displayed on the external device 108. The display screen 1600 includes information 1602 indicating that the patient is now ready for the MRI scan to be conducted and that a user selectable control 1606 should be selected once the MRI scan is complete.

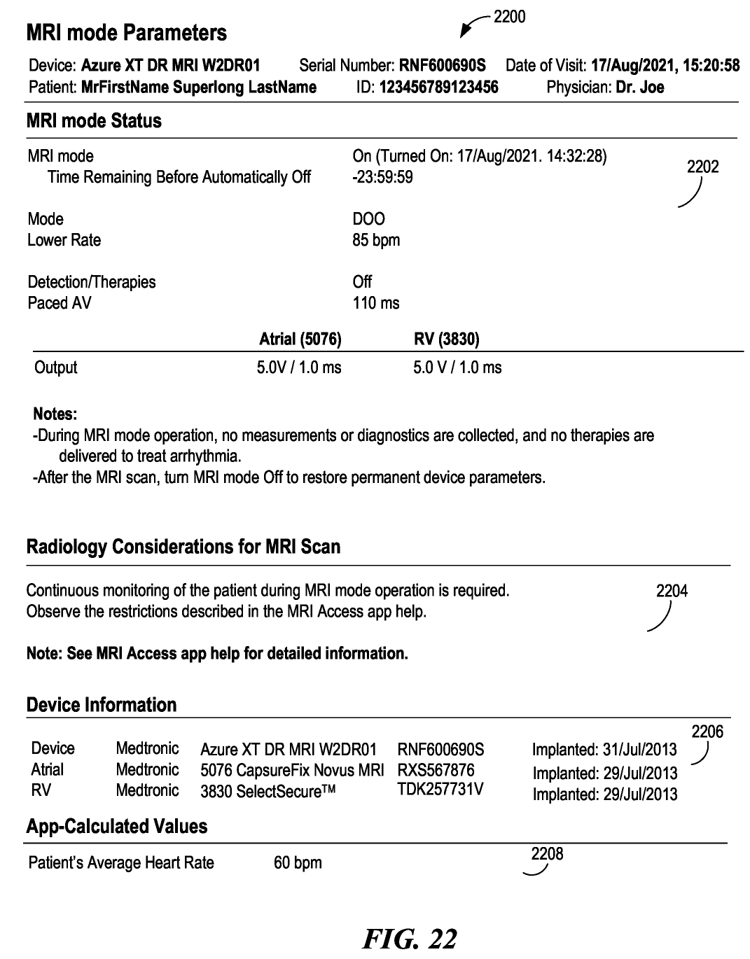

The display screen 1600 includes additional features such as a user selectable control 1604 that allows the MRI mode parameters report of FIG. 22 to be generated. The connection status indicator 1608 shows the connection of the external device 108 to only the connector device 112 and not to the implanted medical device 102. The procedural progression timeline 1610 is also present to show that the implanted medical device 102 has been checked at stage 1612, the MRI mode is activated at stage 1614, with the device now ready for the MRI scan at stage 1616, and with no deactivation of the MRI mode at stage 1618 since the MRI scan has not yet been performed.

At this point, the MRI technologist or other user 106 can have the patient enter the room of the MRI machine 104 and then load the patient into the MRI machine. The MRI technologist or other user then conducts the MRI scan in accordance with the MRI conditionality of the implanted medical device 102. Once the MRI scan is complete, the MRI technologist or other user then selects the control 1606 at operation 246.

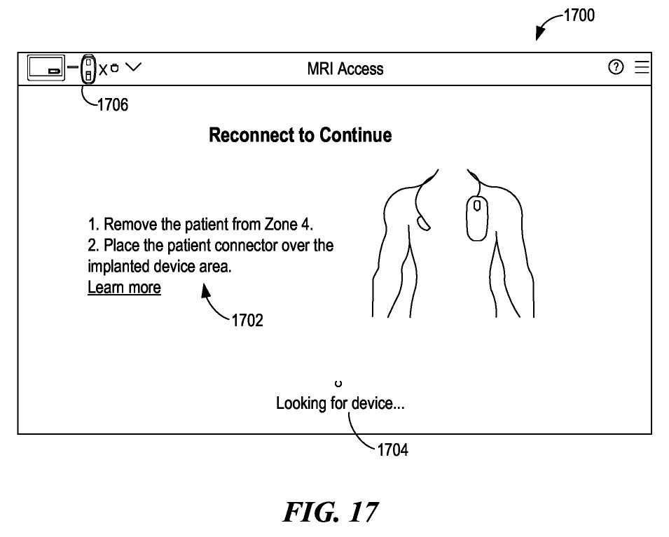

Upon selecting control 1606 at operation 246, the process continues to operation 248 which results in a display screen 1700 of FIG. 17 being displayed at the external device 108. The display screen 1700 includes information 1702 indicating that the MRI technologist or other user should first remove the patient from the room of the MRI machine 104 and then place the connector device 112 at the implanted medical device 102 to reconnect the external device 108 to the implanted medical device 102. A connection progress indication 1704 is provided to indicate that the MRI mode control application is looking for the implanted medical device 102 while the MRI technologist or other user 106 is attempting to re-establish the connection using the connection device 112. The connection status indicator 1706 shows that the external device is connected only to the connector device 112 and not yet to the implanted medical device 102.

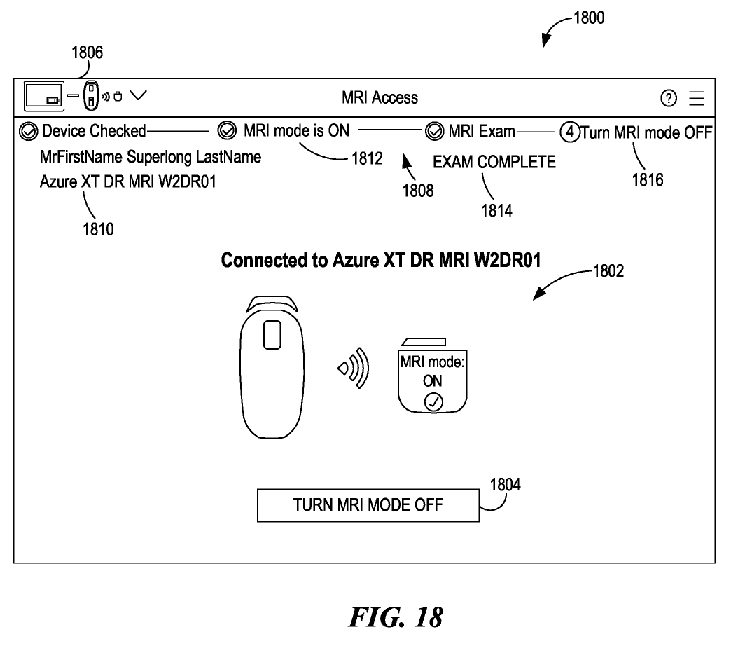

The MRI technologist or other user places the connection device 112 at the implanted medical device 102 once the patient is out of the room where the MRI machine 104 is located and this results in reconnection of the external device 108 to the implanted medical device 102 at operation 250. The process then continues to operation 252 which results in the display of a display screen 1800 of FIG. 18. The display screen 1800 includes information 1802 indicating that the reconnection to the implanted medical device 102 has occurred. The display screen 1800 also includes a user selectable control 1804 that allows the MRI technologist or other user 106 to deactivate the MRI mode of the implanted medical device 102 in order to return the implanted medical device back to the normal mode of operation.

The display screen 1800 includes additional features such as the connection status indicator 1806 that shows the connection of the external device 108 to both the connector device 112 and to the implanted medical device 102. The procedural progression timeline 1808 is also present to show that the implanted medical device 102 has been checked at stage 1810, the MRI mode is activated at stage 1812, with the MRI scan now complete at stage 1814, and deactivation of the MRI mode now being available at stage 1816.

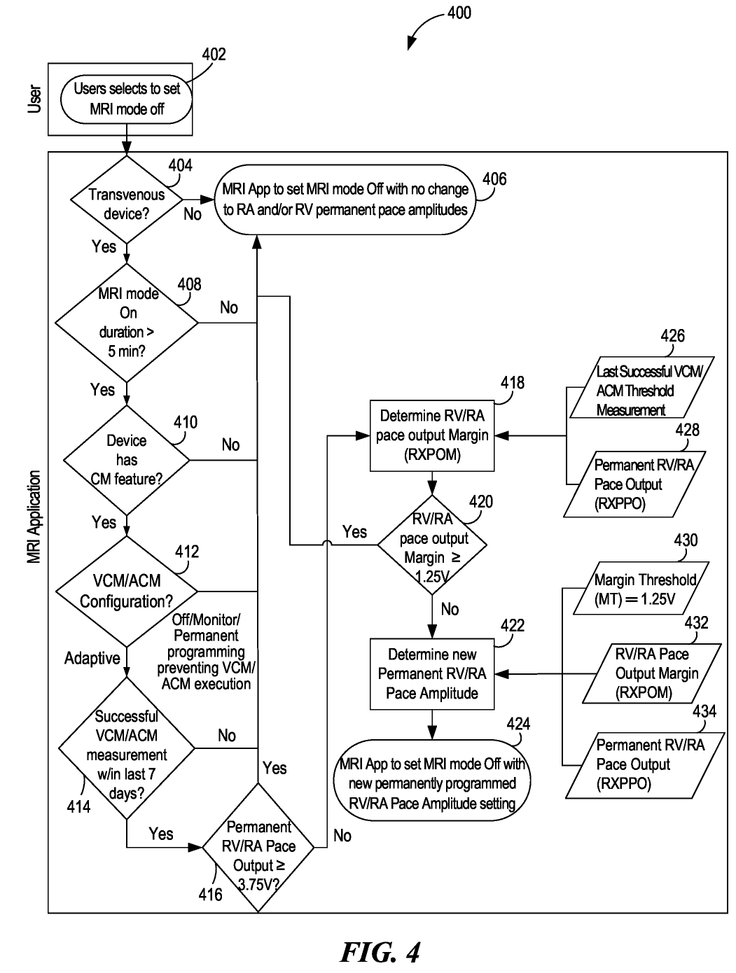

Upon selection of the control 1804 at operation 254, the process continues to the operation 256. Here, the MRI mode control application sends an instruction to the implanted medical device 102 to cease operating in the MRI mode by deactivating the MRI mode and returning to the normal mode of operation. For patients with implanted cardiac pacing devices, the MRI mode control application may also initiate a post-scan pace output algorithm without any further input from the user, so the MRI technologist or other user 106 is not required to separately initiate the post-scan pace output algorithm. This post-scan pace output algorithm, which is discussed in more detail below with reference to FIG. 4 for an example where the implanted medical device 102 has a cardiac function, sets the pacing amplitude for the normal mode of operation that occurs once the MRI mode is deactivated.

The post-scan pace output algorithm may be performed in one of various places. In one scenario, the post-scan pace output algorithm is performed at the external device 108 and the result is then programmed into the implanted medical device 102. In another scenario, the implanted medical device 102 is configured to implement the post-scan pace output algorithm upon receiving an instruction to deactivate the MRI mode.

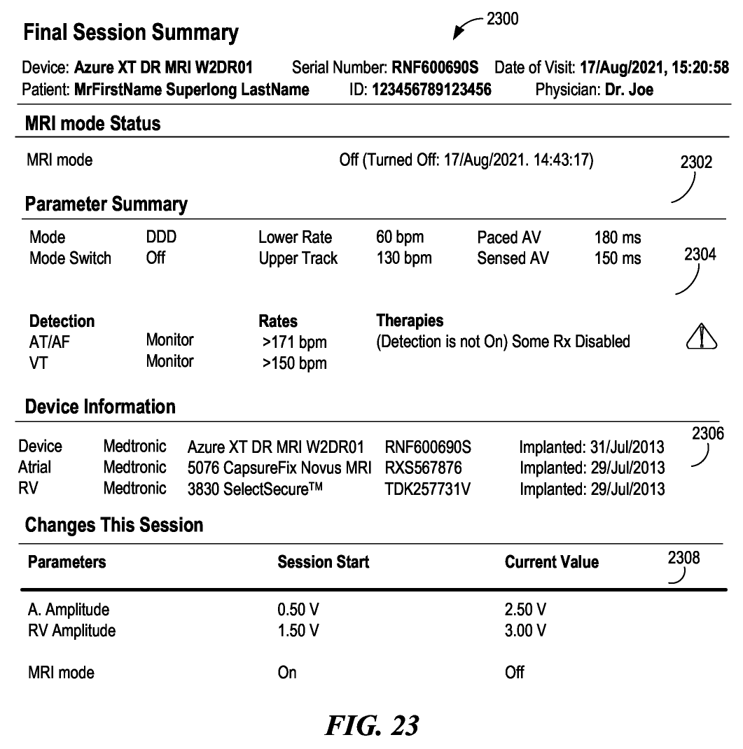

Once the MRI mode control application has completed the post-scan algorithm, the process continues to operation 258 and termination state 260 where the implanted medical device reports that the normal mode of operation is active instead of the MRI mode. Here, a display of a display screen 1900 occurs at the external device 108. The display screen 1900 includes information 1902 indicating that the connector device 112 may be removed from the area of the implanted medical device 102 and that a post-scan report is available. The display screen 1900 also includes a user selectable control 1904 that causes the post-scan device report, such as the post-scan device report of FIG. 23, to be generated. A user selectable control 1906 is provided to allow the MRI technologist or other user 106 to close out the connection to the implanted medical device 102 to end the process.

The display screen 1900 includes additional features such as the connection status indicator 1908 that shows the connection of the external device 108 to both the connector device 112 and to the implanted medical device 102. The procedural progression timeline 1910 is also present to show that the implanted medical device 102 has been checked at stage 1912, the MRI mode was previously activated at stage 1914, with the MRI scan now complete at stage 1916, and the MRI mode now being deactivated at stage 1918.

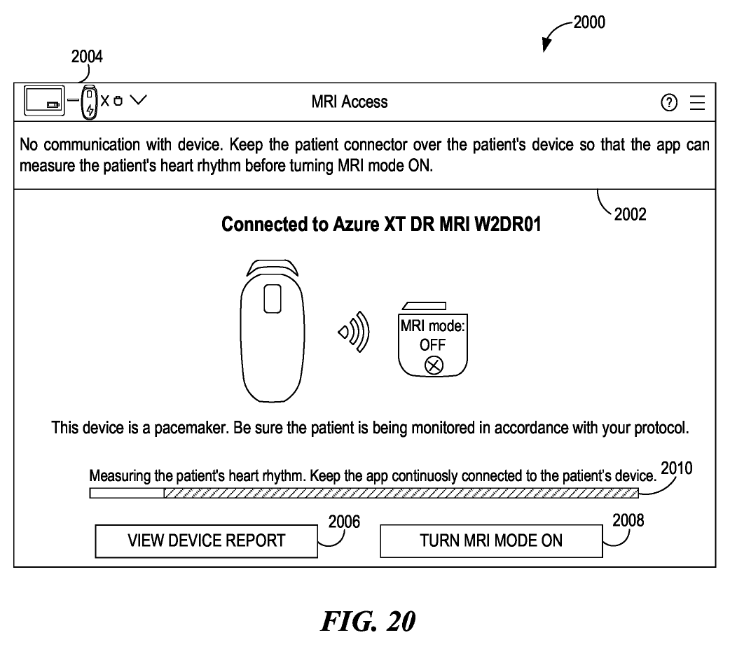

FIG. 20 shows a display screen 2000 that may be displayed at the external device 108 if the connection to the implanted medical device 102 is lost at any time before the MRI mode has been activated at operation 236. This corresponds to the period of time when the MRI mode control application is monitoring the heart rhythm of the patient in order to verify that there is no cardiac arrhythmia and to perform the mode and rate determinations. The display screen 2000 includes information to the MRI technologist or other user 106 regarding keeping the connector device 112 over the implanted medical device 102 to maintain the connection.

The display screen 2000 includes additional features. The connections status indicator 2004 shows that the external device 108 is only connected to the connector device 112 and not to the implanted medical device 102. A user selectable control 2006 allows the MRI technologist or other user 106 to generate the pre-scan report of FIG. 21. A user selectable control 2008 to activate the MRI mode is inactive and not selectable at this point since the connection has been lost with the implanted medical device 102. A heart rhythm monitoring progress bar 2010 is displayed to indicate to the MRI technologist or other user 106 that the heart rhythm monitoring needs to continue in order to be adequate for conducting the necessary pre-MRI mode activation checks that ultimately cause the control 2008 to become active.

As discussed above in relation to operation 216, the MRI mode control application of the external device 108 may start the heart tracker module in the background. One example 300 of the operations of the heart tracker module are shown in FIGS. 3A and 3B. The heart rate tracker module monitors the telemetry connection to the implanted medical device 102 through the connector device 112 and maintains the current heart rate queue that is used as a therapy parameter when deciding whether to allow activation of the MRI mode. The heart rate tracker module 300 therefore also controls whether the user interface controls such as the user selectable control for activating the MRI mode 1306 of FIG. 13 are enabled for display and selection by the MRI technologist or other user 106 based on the heart rate interval queue.

At an operation 302, the operations begin from the point of a last session interrogation where query 306 of an initialization submodule 304 detects whether a flashback of heart rhythm data exists in device memory and is valid. If not, then operation 308 initializes an empty heart rate interval queue and the initialization is complete. If a valid flashback does exist, then operation 310 takes the recent 30 seconds of available flashback events and query 312 detects from the 30 seconds of flashback events whether the pacing mode is AOO, AAI, or AAIR. If one of those pacing modes was used, then operation 314 takes valid events having an atrial-atrial (A-A) interval, then the process proceeds to operation 316. If none of those pacing nodes were used as determined at query 312, then operation 318 takes valid events having a ventricular-ventricular (V-V) interval and the process continues to operation 316. At operation 316, the heart rate interval queue is prefilled with the flashback events so that there is already data in the heart rate interval queue for subsequent consideration.

At this point, multiple activities may occur contemporaneously including a telemetry handler 320, a graphical user interface (GUI) callback handler 340, a GUI handler 346, and a heart rate interval supplemental marker tracker submodule 354. The telemetry handler 320 monitors the telemetry connection to the device connector 112 and implanted medical device 102 in order to manage the heart rate queue. A query 322 determines whether telemetry has been lost. If not, then process loops back to the query 322 to continuously determine if the telemetry has been lost. If the query 322 detects that the telemetry has been lost, then an operation 324 starts a timer to measure the length of time the connection has been lost.

The process may take different paths depending on the timer and whether communications are restored. A query 326 detects whether communication with the implanted medical device has become activate again. If so, then operation 328 resets the timer and the process returns to the query operation 322. If communication has not become active, then query 330 detects whether the timer has reached a threshold, which is 300 seconds in this example but could be a smaller or larger time in other examples. If the threshold has not been reached, then the process loops back to query operation 326 to again determine if communications are active again. If the threshold has been reached before communications become active again, then operation 332 initializes an empty heart rate interval queue to begin the process of collecting fresh 30 second spans of heart rate data to the heart rate interval queue.

While the telemetry handler is operating to watch for communication time outs, the heart rate interval supplemental marker tracker submodule 354 is also operating to monitor new heart rate interval markers being communicated by the implanted medical device 102 to the external device 108 and will add the received values to the heart rate interval queue. Initially, a query 356 detects whether the pacing mode is currently an atrial pacing mode such as AOO, AII, or AAIR. If so, then query 358 monitors for A-A interval supplemental markers. If 30 seconds of A-A intervals have been collected, then operation 360 updates the heart rate interval queue with those 30 seconds of A-A interval supplemental markers. If not, collection of the A-A interval supplemental markers continues until the amount collected reaches 30 seconds. This process repeats to continually keep a fresh heart rate interval queue of the last 30 seconds of A-A intervals detected by the implanted medical device 102.

For ventricular pacing modes (modes other than AOO, AAI, AAIR), V-V intervals will be monitored by query 362. Once 30 seconds of V-V interval supplemental markers have been collected, then operation 364 updates the heart rate interval queue with those 30 seconds of V-V interval supplemental markers. If not, collection of the V-V interval supplemental markers continues until the amount collected reaches 30 seconds. This process repeats to continually keep a fresh heart rate interval queue of the last 30 seconds of V-V intervals detected by the implanted medical device 102. It will be appreciated that the 30 seconds threshold of this example may be smaller or longer in other examples, but 30 seconds of A-A or V-V interval supplemental markers are considered adequate for determining the patient’s average heart rate and allowing the MRI technologist or other user 106 to enable the MRI mode.

While the telemetry handler 320 and the heart rate interval submodule 354 are operating, a GUI handler 346 is also operating to control whether the user selectable controls are made available to the MRI technologist or other user 106 for purposes of activating the MRI mode. The GUI handler 346 may determine whether the user selectable control 1306 of FIG. 13 is enabled for selection based upon an analysis of the heart rate interval data that has been acquired in the heart rate interval data queue by the initialization module 304 and the heart rate interval supplemental tracker submodule 354.

At a query 348 the GUI handler 346 determines whether the sum of the sampled intervals in the heart rate interval queue are greater than or equal to a threshold which in this example is 30 seconds. If the sum of the sampled intervals is less than the threshold, then operation 350 maintains the user selectable control 1306 for activating the MRI mode is in a deactivated state so that it cannot be selected by the MRI technologist or other user 106 even when the display screen 1300 is present on the display of the external device. If the sum of the sampled intervals in the heart rate interval queue are greater than 30 seconds, then this constraint is satisfied so that the user selectable control 1306 is enabled for selection at operation 352. The process continues by returning to the query 348.

While the telemetry handler 320, the GUI handler 346, and the heart rate interval submodule 354 are operating, the GUI callback handler 340 is also operating to detect the selection of the user selectable control 1306 to activate the MRI mode. A query 342 awaits the selection of control 1306 by the MRI technologist or other user 106 to activate the MRI mode. Once the selection is made, operation 344 determines the current heart rate from the sampled intervals in the heart rate interval queue so that the rate and mode algorithm referred to above with reference to operation 236 of FIG. 2 has a most current measure of the heart rate.

The heart rate may be expressed in various ways in the heart rate interval queue but beats per minute is a conventional unit of measure that may be expressed to the MRI technologist or other user 106, in the pre- and post-scan device reports of FIGS. 21 and 22, and as used in the rate/mode algorithm used to configure the MRI mode. To the extent necessary, the operation 344 may convert the heart rate unit of measure to beats per minute. For example, if the number of A-A or V-V intervals representing beats is stored over a duration measured in milliseconds, then the total number of beats divided by the duration in milliseconds is multiplied by a conversion factor of 60,000 milliseconds per minute to achieve a heart rate in beats per minute.

As discussed above in relation to operation 256, the MRI mode control application of the external device 108 may start the post-scan pace output operations in the background. An example 400 of those post-scan pace output operations are shown in FIG. 4. The post-scan pace output process steps through various determinations in order to decide whether to set new right ventricular and right atrial pace amplitudes.

These operations begin with the state 402 where the MRI technologist or other user has selected user selectable control to deactivate the MRI mode and return the implanted medical device 102 to a normal operating mode. Query operation 404 detects whether the implanted medical device 102 is a transvenous device. This may be done from a look-up of the device identifier in the prior mentioned database to determine the device type. If the implanted medical device 102 is not a transvenous device, then the MRI mode control application deactivates the MRI mode and does not change the existing therapy parameters of the normal mode including the right atrium and right ventricle permanent pace amplitudes in the case of a cardiac device at state 406.

Where the implanted medical device 102 is a transvenous device, query operation 408 then determines whether the MRI mode was activated for a duration greater than a threshold, such as 5 minutes in the example shown. The MRI mode control application knows this duration as the amount of time from sending the instruction to the implanted medical device 102 to enter the MRI mode to the sending of the instruction to the implanted medical device 102 to exit the MRI mode. If the threshold was not exceeded, then the process proceeds to operation 406.

Where the threshold was exceeded as determined at query 408, then query operation 410 determines whether the implanted medical device 102 has a capture management feature which will routinely monitor stimulation thresholds for pacing the patient’s heart. The MRI mode control application detects this either from the information obtained from the implanted medical device 102 or from a look-up of the device identifier. If no capture management feature is present, then the process proceeds to operation 406.

Where the capture management is present, then query operation 412 determines how the ventricular capture management and atrial capture management is configured. The configuration may be in one of the following modes off, monitor, or an adaptive mode of execution. The MRI mode control application detects this from the information obtained from the implanted medical device 102. If the configured VCM/ACM mode is off or monitor, then the process proceeds to operation 406.

Where the configured VCM/ACM mode is adaptive, then query operation 414 determines whether the implanted medical device 102 has had a successful VCM/ACM measurement within a threshold amount of time, such as seven days in the example shown. The MRI mode control application detects this from the information obtained from the implanted medical device 102. If the last successful VCM/ACM measured has not occurred within the threshold amount of time, such as because the data was not available within the threshold due to a device power on reset, then the process proceeds to operation 406.

Where the time threshold was not exceeded as determined at query 414, then query operation 416 determines whether a permanent right ventricle/right atrium (RV/RA) pace output of the implanted medical device 102 is at least as much as a voltage threshold, such as 3.75 volts in the example shown. The MRI mode control application detects this from the information obtained from the implanted medical device 102. If the voltage threshold was met or exceeded, then the process proceeds to operation 406.

Where the voltage threshold was not exceeded as determined at query 416, then operation 418 determines an RV/RA pace output margin (RXPOM). RXPOM is equal to the permanent RV/RA pace output (RXPPO), which is RXPPO data 428 obtained from the implanted medical device 102, minus the last successful CMRX threshold, which is the last successful VCM/ACM threshold measurement data 426 obtained from the implanted medical device.

Once the RXPOM is determined, then query operation 420 detects whether the RV/RA pace output margin meets or exceeds a voltage margin threshold, such as 1.25 volts as shown in this example. If the voltage margin threshold (MT) is exceeded, then the process proceeds to operation 406. If the voltage threshold was not exceeded, then operation 422 determines a new permanent RV/RA pace amplitude. The new permanent RV/RA pace amplitude may be computed by the following equation:

$$\begin{matrix}\text{New RX Pace Amplitude =} \\ \left[\text{ceil} \left(\left(\text{MT-RXPOM}\right)\right) * \left(4\right) / 4\right] + \text{RXPPO}\end{matrix}$$

These values used to compute the new RX pace amplitude include the MT value 430 that is pre-defined for the post-scan output process and stored by the external device 108, the RXPOM data value 432 that is obtained from the implanted medical device 102, and the RXPPO data value 434 which is also obtained from the implanted medical device 102. This new RX pace amplitude is then programmed to the implanted medical device 102 once the MRI mode is deactivated at the state 424.

As discussed above with reference to FIGS. 2A and 2B, the MRI mode control application provides the MRI technologist or other user 106 with the option to generate preand post-scan reports, and a device ready for scan report. FIG. 21 shows an example of the pre-scan report 2100, FIG. 22 shows an example of a device ready for scan report 2200, and FIG. 23 of the post-scan report 2300. These reports include an MRI mode status section 2102, 2202, 2302. The section 2102 shows that the MRI mode is off while the section 2202 shows that the MRI mode is on, with a pacing mode, a lower rate, any detection therapy, and a paced A/V interval. The section 2302 shows that the MRI mode is off again. This information comes from the operations of the external device 108 and the interaction with the implanted medical device 102.

Both pre-scan reports also include a section 2104, 2204 of radiology considerations, and all three reports include a “Device Information” section 2108, 2206, 2306. These sections are the same in all three reports. Sections 2104, 2204 are static information while sections 2108, 2206, 2306 include information either received from interrogating the implanted medical device 102 and/or looking up the device identifier in the aforementioned device database.

The pre-scan report 2100 and the post-scan report 2300 include a “Parameter Summary” section 2106, 2304, respectively, that is not present in the report 2200. This “Parameter summary section 2106 includes at least some of the information that has been obtained from the implanted medical device 102 during the interrogation. Information such as the pacing mode, lower rate, paced A/V and so on are available for inspection. The parameter summary section 2106 can be compared to the parameter summary section 2304 to ensure the implanted device 102 has been returned to the original settings after the MRI mode has been deactivated.

The report 2100 also includes a “Device Status section” 2110 not present in the report 2200 or report 2300. This section 2110 provides information such as the remaining longevity of the device, the lead impedances, capture threshold, and so on for inspection. This information comes from the interrogation of the implanted medical device 102.

The report 2200 includes an “Application Calculated Values” section 2208 that is not included in the report 2100 or the report 2300. The information in this section 2208 includes information calculated by the MRI mode control application. In this example, the section includes the average heart rate that has been calculated using the heart rate tracking operations of FIGS. 3A and 3B.

The report 2300 includes a “Changes This Session” section 2308 that is not included in the report 2100 or the report 2200. The information in this section 2308 shows therapy parameter values at the start of the MRI mode and the current therapy parameters once the MRI mode has been deactivated and the post-scan analysis is complete so they can be easily compared.

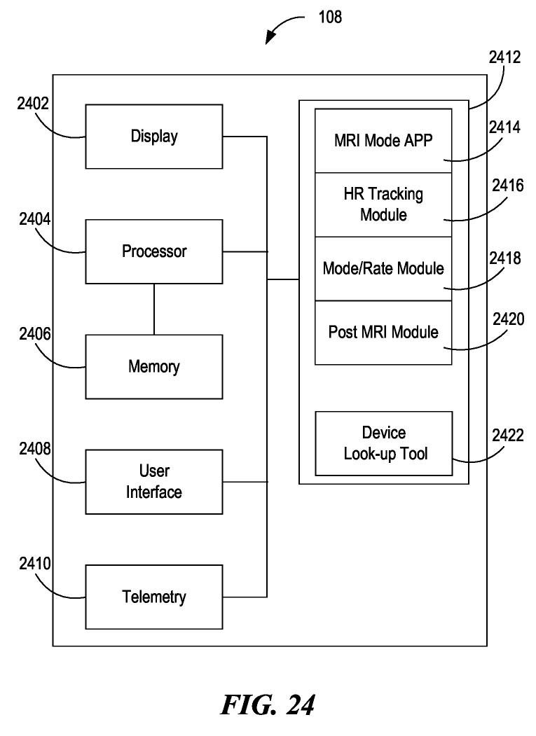

FIG. 24 shows an example of the components of the external device 108. The external device 108 may be a standard off-the-shelf computing device such as a handheld tablet, smartphone, personal computer, and the like or may be a special purpose computing device. In this example, the external device 108 includes a display system 2402 including a display screen and any combination of hardware and software that allows the display to show display screens like those discussed above.

The external device 108 also includes a processing device 2404 to execute the logical operations discussed above in FIG. 2A-4 in order to produce the display screens by instructing the display system, receive the user inputs, and communicate with the other devices. The processing device 2404 may be of many forms such as a general-purpose programmable processor, a dedicated purpose processor, hardwired logic, various combinations, and the like. Operating memory 2406 such as random-access memory, read only memory, flash memory, various combinations, and the like is accessible by the processing device 2404.

A user interface 2408 is provided to allow the user such as an MRI technologist or other user 106 to provide input into the external device 108 that is delivered to the processing device 2404, such as to make selections of the user selectable controls discussed above. The user interface 2408 may take various forms such as a mouse, keyboard, touchscreen, various combinations, and the like.

A wireless telemetry circuit 2410 is provided to establish the longer-range wireless telemetry signals 110 to communicate with the connector device 112, if needed, to ultimately communicate with the implanted medical device 102. The wireless telemetry circuit 2410 may communicate directly with the implanted medical device 102 where the implanted medical device 102 is capable of exchanging the longer-range wireless signals 110. The processing device 2404 sends and receives all information including data and instructions being exchanged with the implanted medical device 102 through the wireless telemetry circuit 2410.

A storage device 2412 is also present to allow the processing device 2404 to access stored applications and other data. In this example, the storage device 2412 includes the MRI mode control application 2414 such as the example 200 shown in FIGS. 2A-2B, the heart rate tracking module 2416 such as the example 300 shown in FIGS. 3A-3B, the mode/rate module 2418 such as the examples disclosed in U.S. Pat. No. 10,293,167 and U.S. Pat. No. 10,441,798, and the post-scan module 2420 such as the example 400 shown in FIG. 4. Additionally, the external device 108 may include a device look-up tool 2422, such as the tool shown in FIG. 5, either in the form of a stand-alone application, a website loaded within a browser application, or a module accessible by the MRI mode control application.

The storage device 2412 may be of various forms, such as a magnetic storage device, solid state device, various combinations, and the like. Furthermore, the storage device 2412 may be of a non-volatile type to avoid the loss of the applications and other data in the event of a loss of electrical power. The external device 108 includes a power source such as a battery or a circuit for receiving power from an external utility and should the power source be removed or otherwise become unavailable, the non-volatile storage 2412 maintains the information.

Thus, it can be appreciated that providing the external device 108 to an MRI technologist or other user 106 allows for the MRI technologist or other user 106 to conduct the screening and preparation of the implanted medical device 102 for an MRI scan. The use of the MRI mode control application 2414 and related modules 2416, 2418, 2420 and the look-up tool 2422 allow the MRI technologist or other user 106 to activate the MRI mode of the implanted medical device 102 and conduct an MRI when doing so is appropriate for the implanted medical device 102 and then deactivate the MRI mode to return to the normal mode of operation.

While embodiments have been particularly shown and described, it will be understood by those skilled in the art that various other changes in the form and details may be made therein without departing from the spirit and scope of the invention.

What is claimed is:

1. A method of preparing an implanted medical device of a patient for a magnetic resonance imaging (MRI) scan, comprising:
wirelessly communicating from an external device to the implanted medical device to begin receiving current therapy and device parameters;
determining by the external device whether the therapy parameters and device parameters are within acceptable ranges for the MRI scan;
when the therapy parameters and device parameters are within acceptable ranges for the MRI scan, displaying a first user selectable control at the external device to implement an MRI mode of operation at the implanted medical device; and
when one or more of either the therapy parameters or the device parameters are outside of the acceptable ranges for the MRI scan, displaying a message at the external device regarding not conducting the MRI scan and not displaying the first user selectable control to implement the MRI mode of operation.

2. The method of claim 1, wherein wirelessly communicating from the external device to the implanted medical device comprises communicating wirelessly between the external device and a connector device that communicates wirelessly with the implanted medical device.

3. The method of claim 2, further comprising:
prior to wirelessly communicating from the external device to the implanted medical device, displaying a message at the external device to instruct the user to wirelessly connect the connector device with the external device;
detecting the connection to the connector device at the external device;
after detecting the connection to the connector device, displaying at the external device an instruction to place the connector device relative to the implanted medical device;
after the connector device has been placed relative to the implanted medical device, interrogating by the external device the implanted medical device to begin wirelessly receiving the current therapy and device parameters.

4. The method of claim 1, further comprising:
receiving a selection of the first user selectable control by a user at the external device; and
in response to receiving the selection of the first user selectable control, sending an instruction from the external device to the implanted medical device to begin operating in the MRI mode.

5. The method of claim 4, wherein in response to receiving the selection of the first user selectable control, initiating by the external device a determination of therapy parameters for the MRI mode.

6. The method of claim 5, wherein the external device determines the therapy parameters for the MRI mode and sends the therapy parameters for the MRI mode to the implanted medical device.

7. The method of claim 4, further comprising:
after sending the instruction, displaying at the external device a second user selectable control to acknowledge that the MRI scan is complete;
receiving a selection of the second user selectable control by the user at the external device; and
after receiving the selection of the second user selectable control, displaying at the external device a third user control to terminate the MRI mode at the implanted medical device.

8. The method of claim 7, wherein wirelessly communicating from the external device to the implanted medical device comprises communicating wirelessly between the external device and a connector device that communicates wirelessly with the implanted medical device, the method further comprising:
after receiving the selection of the second user selectable control and prior to displaying the third user selectable control, displaying at the external device a notice to position the connector device at the implanted medical device to reconnect to the implanted medical device, and after to reconnecting to the implanted medical device displaying at the external device the third user control.

9. The method of claim 7, further comprising:
receiving a selection of the third user selectable control by the user at the external device; and
in response to receiving the selection of the third user selectable control, sending an instruction from the external device to the implanted medical device to cease operating in the MRI mode.

10. The method of 4, further comprising:
displaying at the external device a prompt for the user to determine if a patient with the implanted medical device is comfortable after the instruction is sent to the implanted medical device to begin operating in the MRI mode;
displaying at the external device a fourth user selectable control to indicate whether the patient is feeling comfortable;
receiving a selection of the fourth user selectable control;
when the selection of the fourth user selectable control indicates that the patient is not comfortable, then displaying at the external device a third user control to terminate the MRI mode at the implanted medical device;
receiving a selection of the third user selectable control by the user at the external device; and
in response to receiving the selection of the third user selectable control, sending an instruction from the external device to the implanted medical device to cease operating in the MRI mode.

11. The method of claim 4, wherein the implanted medical device provides a cardiac pacing function, the method further comprising:
after sending the instruction from the external device to the implanted medical device to begin operating in the MRI mode, sending an instruction to the implanted medical device to cease operating in the MRI mode; and
after sending the instruction to the implanted medical device to cease the MRI mode, then initiating by the external device without further user input a post-scan analysis to determine an appropriate therapy parameter to be provided by the implanted medical device upon ceasing the MRI mode and providing the appropriate therapy parameter determined by the post-scan analysis to the implanted device.

12. The method of claim 1, further comprising:
prior to wirelessly communicating from the external device to the implanted medical device, displaying a message to the external device to confirm that the implanted medical device is MRI compatible;
providing a fifth user selectable control to indicate that the user has confirmed that the implanted medical device is MRI compatible;
receiving a selection of the fifth user selectable control at the external device; and
in response to receiving the selection of the fifth user selectable control, beginning to wirelessly communicate from the external device to the implanted medical device.

13. The method of claim 1, further comprising:
prior to displaying the first user selectable control at the external device to implement an MRI mode of operation at the implanted medical device, determining at the external device whether the implanted medical device is already in the MRI mode;
when the implanted medical device is already in the MRI mode, then displaying at the external device an inquiry as to whether the MRI mode should be deactivated with a sixth user selectable control to turn off the MRI mode and a seventh user selectable control to maintain the MRI mode and continue with the MRI scan; and
when the implanted medical device is not already in the MRI mode, then displaying the first user selectable control at the external device.

14. The method of claim 13, when receiving a selection of the sixth user selectable control, then sending an instruction from the external device to the implanted medical device to cease the MRI mode and when receiving a selection of the seventh user selectable control, then displaying at the external device a second user selectable control to indicate that the MRI scan is complete.

15. The method of claim 1, wherein the external device receives a device identifier from the implanted medical device, the method further comprising determining at the external device whether a function of the external device for starting MRI mode is compatible with the implanted medical device and when the function of the external device is not compatible with the implanted medical device, then displaying at the external device that the implanted medical device is not compatible and not displaying the first user selectable control.

16. An external device for preparing an implanted medical device of a patient for a magnetic resonance imaging (MRI) scan, comprising:
a display;
a user interface;
a wireless telemetry circuit;
a processing device in communication with the display, the user interface, and the wireless telemetry circuit, the processing device being configured to:
wirelessly communicate with the implanted medical device to begin receiving current therapy and device parameters by using the telemetry circuit;
determine whether the therapy parameters and device parameters are within acceptable ranges for the MRI scan;
when the therapy parameters and device parameters are within acceptable ranges for the MRI scan, display a first user selectable control on the display to implement an MRI mode of operation at the implanted medical device, the first user selectable control being selectable via the user interface; and
when one or more of either the therapy parameters or the device parameters are outside of the acceptable ranges for the MRI scan, displaying a message on the display regarding not conducting the MRI scan and not displaying the first user selectable control to implement the MRI mode of operation.

17. The external device of claim 16, wherein wirelessly communicating with the implanted medical device comprises communicating wirelessly between the telemetry circuit and a connector device that communicates wirelessly with the implanted medical device.

18. The external device of claim 16, wherein the processing device is further configured to:
receive a selection of the first user selectable control by a user through the user interface; and
send an instruction to the implanted medical device to begin operating in the MRI mode.

19. The external device of claim 18, wherein in response to receiving the selection of the first user selectable control, initiate a determination of therapy parameters for the MRI mode from the current therapy parameters.

20. The external device of claim 19, wherein the processing device determines the therapy parameters for the MRI mode and sends the therapy parameters for the MRI mode to the implanted medical device through the telemetry circuit.

21. The external device of claim 19, wherein the processing device is further configured to:
after sending the instruction, display a second user selectable control to acknowledge that the MRI scan is complete;
receive a selection of the second user selectable control by the user through the user interface; and
after receiving the selection of the second user selectable control, display a third user control to terminate the MRI mode at the implanted medical device.

22. A system, comprising:
an implanted medical device;
an external device for preparing the implanted medical device of a patient for a magnetic resonance imaging (MRI) scan, comprising:
a display;
a user interface;
a wireless telemetry circuit;
a processing device in communication with the display, the user interface, and the wireless telemetry circuit, the processing device being configured to:
wirelessly communicate with the implanted medical device to begin receiving current therapy and device parameters by using the telemetry circuit;
determine whether the therapy parameters and device parameters are within acceptable ranges for the MRI scan;
when the therapy parameters and device parameters are within acceptable ranges for the MRI scan, display a first user selectable control on the display to implement an MRI mode of operation at the implanted medical device, the first user selectable control being selectable via the user interface; and
when one or more of either the therapy parameters or the device parameters are outside of the acceptable ranges for the MRI scan, displaying a message on the display regarding not conducting the MRI scan and not displaying the first user selectable control to implement the MRI mode of operation.

23. The system of claim 22, further comprising a connector device, and wherein wirelessly communicating with the implanted medical device comprises communicating wirelessly between the telemetry circuit and the connector device that communicates wirelessly with the implanted medical device.

24. The system of claim 22, wherein the processing device is further configured to:
receive a selection of the first user selectable control by a user through the user interface; and
send an instruction to the implanted medical device to begin operating in the MRI mode.

25. The system of claim 24, wherein in response to receiving the selection of the first user selectable control, initiate a determination of therapy parameters for the MRI mode from the current therapy parameters.

26. The system of claim 25, wherein the processing device determines the therapy parameters for the MRI mode and sends the therapy parameters for the MRI mode to the implanted medical device through the telemetry circuit.

27. The system of claim 25, wherein the processing device is further configured to:
after sending the instruction, display a second user selectable control to indicate that the MRI scan is complete;
receive a selection of the second user selectable control by the user through the user interface; and
after receiving the selection of the second user selectable control, display a third user control to terminate the MRI mode at the implanted medical device.
